(19) United States
(12) Patent Application Publication
(10) Pub. No.: US 2016/0055305 A1
(43) Pub. Date: Feb. 25, 2016

(54) VIDEO ENHANCEMENTS FOR LIVE SHARING OF MEDICAL IMAGES

(71) Applicant: eagleyemed, Inc., Santa Clara, CA (US)

(72) Inventors: Harish P. HIRIYANNAIAH, San Jose, CA (US); Muhammad Zafar Javed SHAHID, San Jose, CA (US)

(21) Appl. No.: 14/463,127

(22) Filed: Aug. 19, 2014

(51) Int. Cl. G06F 19/00; H04L 29/06; H04N 19/593; H04N 21/647; A61B 5/00; H04N 7/15

(52) U.S. Cl. CPC G06F 19/3418; A61B 5/0013; H04N 7/15; H04N 19/593; H04N 21/64792; H04N 21/64769; H04L 65/607

(57) ABSTRACT

In a telemedicine application there is live sharing of a video stream of medical images from a first site to a second site as well as a two-way conferencing capability. Live streaming of medical images in a live interactive session imposes many limitations on the video streaming process not found in conventional video conferencing. The network conditions are heterogeneous and low latency is required to support: 1) live streaming of medical images to a remote site and 2) supporting two-way conferencing in which a doctor or clinician at the remote site can provide real-time analysis or guidance on how to adjust a location of an imaging device. A suite of video enhancements is disclosed to improve the capability to sustain live video streaming of medical images in a telemedicine environment including a two-way conference between doctors or clinicians.

### FIELD OF THE INVENTION

The present invention is generally related to video enhancements for low latency video applications across heterogeneous network conditions. More particularly, the present invention is directed towards enhancing a low latency video application for telemedicine application in which a live video stream of medical images is shared.

### BACKGROUND OF THE INVENTION

In some telemedicine applications there are a number of strict requirements imposed when streaming live medical video between a sending node and a receiving node.

First, in many telemedicine applications a live video stream of medical video images is transmitted to support a live conference between medical professionals. As a result there is a very tight latency requirement in order to support live streaming. In particular, in many telemedicine applications there is a full duplex communication session in which latency is a bottleneck to maintaining a live video stream of medical images in duplex communication sessions. For example, in the context of ultrasound imaging, an ultrasound technician may be located at first location and a radiologist may be located at a second location. In a live session between the ultrasound technician and the radiologist, the technician needs input from the radiologist as to the direction the ultrasound technician should move the ultrasound probe. That is, the radiologist sees an ultrasound image, analyzes the image, and gives instructions for the technician to move the probe to a new location. This live, interactive session creates a tight latency requirement to support an interactive live session that creates a good user-experience for the radiologist and the ultrasound technician to work together as a team. The tight latency requirement makes it impractical in many applications to employ packet retransmission to deal with lost or corrupted data packet. That is, because the latency requirements are very strict, a result is that it becomes impossible to detect lost/corrupted packets, request retransmission, and receive the retransmitted packets fast enough to support a live video stream.

Second, in many telemedicine applications the network conditions between two sites can vary widely. For example, one of the sites may be at a location with a poor connection to the Internet, such as at a remote location with a wireless Internet connection. Additionally, in many parts of the world local clinics share a network connection with a number of doctors and clinicians such that bandwidth per user may vary depending on the number of active users at a particular network site. Packet loss and congestion can also be dependent on network conditions.

Third, many conventional approaches to dealing with packet loss in video conferencing cannot be employed for live streaming videos of medical images. In telemedicine applications video post-processing and pre-filtering are generally not employed because of the need to avoid showing false data in medical images. As an example, video image post-processing techniques used in video teleconferencing typically employ smoothing algorithms to deal with lost or corrupted data, such as filling in missing pixels based on information from spatio-temporal surrounding pixels (or neighboring pixels). This is often adequate in the context of sending images of people during a video conference as the smoothing out has no down-side risks. However, in medical images, such smoothing could result in a false diagnosis. For example, if data is corrupted or missing for a pixel of an unhealthy region of a patient, post-filtering techniques that smooth out that region may result in giving a false indication that the tissue is healthy. Additionally, some medical video streams, such as ultrasound images, have a high entropy content, which makes it difficult to effectively perform lossless pre-filtering.

Thus, in a telemedicine application with a low latency requirement it is often not practical to request duplicate packets and this problem is exacerbated because it is also not possible to perform video post-processing to fill in data for missing packets. If a video packet is either late or lost, the entire slice is lost for that frame.

A further complication arises when transmitting a live stream of medical images having a high entropy content. In medical imaging, ultrasonic images have a high entropy content and are very dynamic and noisy. As a result the frame-to-frame predictability is poor. For example, if the frames are transmitted by the MPEG-4 standard then the frames are transmitted in a sequence having a reference frame and difference data for following frames (I frames, B frames, and P frames). However, note that the loss of a slice for an I frame results in a prediction error in the P frames that follow it. That is, the low predictability of a high entropy content medical image makes the decoding more sensitive to the loss of I-frame data than conventional video conferencing.

Therefore the present invention was developed in view of these problems associated with live streaming of medical images in a telemedicine environment.

### SUMMARY OF THE INVENTION

In a telemedicine application there is live sharing of a video stream of medical images from a first site to a second site. Live streaming of medical images in a duplex session imposes many limitations on the video streaming process not found in conventional video conferencing, particularly for high entropy content medical images, such as ultrasound images.

A suite of video enhancements is disclosed to improve the capability to sustain live video streaming of medical images in a telemedicine environment having a two-way conference between doctors or clinicians. The individual units in the suite may be used separately, together, or in sub-combinations. A periodic movement multiple reference unit may be selectively used for high entropy content medical images having a periodic biological movement, such as a movement associated with the circulatory system. The number of reference frames may also be selected based on the biological rhythm. A network aware rate control unit monitors network conditions in a feedback path from a receiver to a sender and adapts a video encoding rate at the sender. An adaptive intra refresh unit adapts an intra refresh frequency based on the video content and network conditions. An n-interleaved vertical intra refresh unit reduces peak bandwidth requirement by horizontally interleaving the vertical intra refresh macroblocks over a greater number of frames with a refresh period.

The video enhancements may be implemented as an apparatus on a computer system, as methods, or stored as computer code on a non-transitory computer readable storage medium.

### BRIEF DESCRIPTION OF THE DRAWINGS

FIG. 1 illustrates a system in accordance with an embodiment of the present invention.

FIG. 2 illustrates a method of setting an adaptive intra refresh frequency in accordance with an embodiment of the present invention.

FIG. 3 illustrates a method of performing network aware rate control in accordance with an embodiment of the present invention.

FIG. 4 illustrates a feedback path for performing network aware rate control in accordance with an embodiment of the present invention.

FIGS. 5 and 6 illustrates aspects of utilizing standard deviation of a received bitrate to determine channel properties in accordance with an embodiment of the present invention.

FIG. 7 illustrates an example of vertical intra macroblocks of a frame.

FIG. 8 illustrates bandwidth consideration associated with transmitting vertical intra macroblocks.

FIG. 9 illustrate bandwidth improvement by horizontal interleaving by n of intra macroblocks in accordance with an embodiment of the present invention.

### DETAILED DESCRIPTION

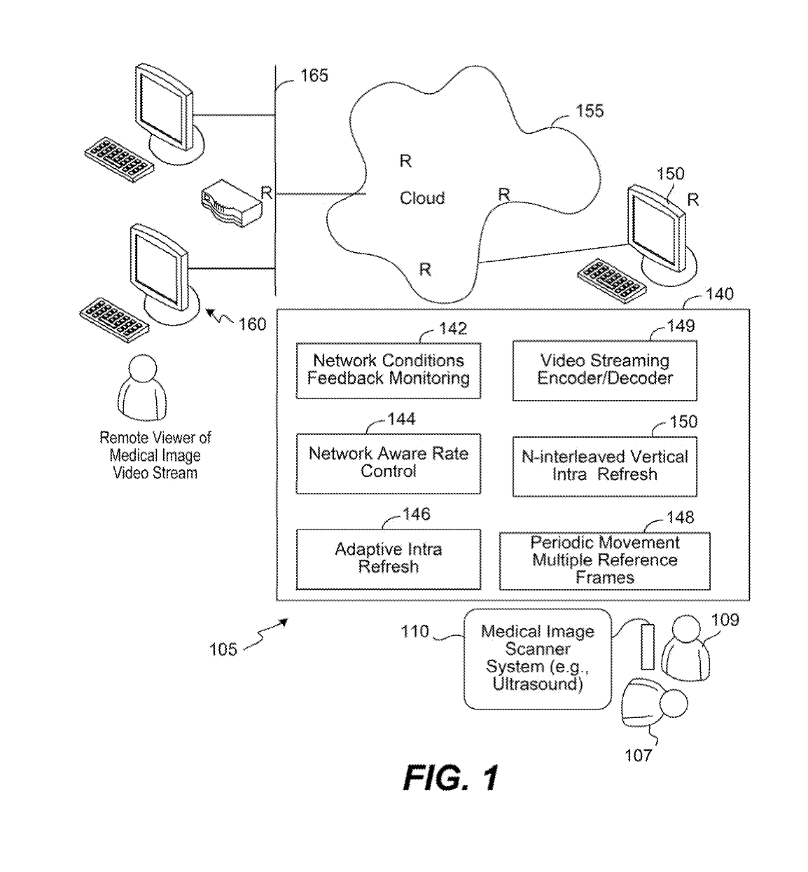

FIG. 1 illustrates an exemplary system and network environment for sharing a video stream of medical images. At a local clinic site 105 a patient 107 is examined by a doctor 109 or a medical technician. The patient may by a human patient. Alternatively, many medical imaging procedures have been adapted for veterinary medicine such that the patient may be cat, dog, horse, etc. A medical imaging scanning device 110 generates a live stream of video images that are transmitted over a network to another site 160, such as local area network 165 of a medical center. As an illustrative example, the live stream may be transmitted to a site of a specialist doctor or a doctor from whom a second opinion is desired. It is also understood that the live stream may also be transmitted simultaneously to other sites.

An exemplary medical imaging scanning device 110 is an ultrasound imaging device, although more generally other types of live imaging device could be used, such as angiography or endoscopy. For the case of ultrasound there is high entropy content of the images in the video stream which in turn invokes many tradeoffs in regards to the compression parameters used to compress the images. Exemplary imaging technologies may require frame rates of 10-60 fps, 8 bits per pixel gray scale and 12 bits for color images, such as color Doppler ultrasound images. In the case of ultrasound imaging, image frames may have a resolution of 512×512 pixels at frame rates of 30 fps and 8 bits per pixel, the raw data rate is 63 Mbps. Other medical imaging techniques, such as angiography, have similar data requirements.

The network path to a remote viewer at site 160 includes the Internet network cloud 155 and any local networks, such as local network 165. Reporting (R) tools are network agents that provide network metrics at different part of the network communication path. Typically there would be reporting tools configured in at least both ends of the network path. These network metrics may include attributes such as bandwidth, packet loss, and packet corruption. The reporting tools may comprise commercial or proprietary reporting tools. The frequency with which reports are received may be configured. For example, many commercial network reporting tools permit periodic generation of reports on network conditions such as once every 100 ms, once every second, once every five seconds, etc.

The network quality of service (QOS) metrics are monitored and used to predict network conditions (in the near future) to determine optimum parameters for transmitting a live video stream of medical images to the remote viewer. That is, the QOS metrics provide metrics on past and recent network conditions, which are then used to predict network conditions when a frame of the live video stream is transmitted.

The network path is heterogeneous. That is, the network path for a session between the local site and the remote site may include several different network portions and the network quality may vary with many different factors such as time of day, number of users on a particular network, and other conditions such as interference (for wireless network portions), and congestion. A live duplex (two-way) video link is supported for doctors and clinicians to share a live video stream of medical images in real time and discuss the images in a live session. Consequently, low latency is required.

A local computer 150 includes a processor and a memory. The local computer 150 includes software modules in block 140 that are used to enhance the operation of a video streaming encoder/decoder application 149 that includes video encoder/decoder modules. The video streaming application 149 may, for example, support a video codec and compression engine generally compliant with a standard such as MPEG-4 or H.264 or other suitable video standard or proprietary format. To support duplex communication, it will be understood that compatible corresponding video encoder/decoder modules may be located at a receiving node, such as at remote site 160.

The video compression may include the use of I-frames (intra-coded pictures), P-frames (prediction picture), and B-frames (bi-predictive pictures). Frames may also be segmented into macroblocks. Where an I-frame has only intra-macroblocks, a P-frame has either intra macroblocks or predicted macroblocks, and B-frames can contain intra, predicted, or b-predicted macroblocks. In the H.264 standard a slice is a distinct region of a frame that is encoded separately from other regions of a frame.

In one embodiment a network conditions feedback monitoring module 142 provides feedback on network conditions, which may be based on the R reporting tools at the receiving site 160 (along with any intermediate reporting locations). A network aware rate control module 144 senses changes in bandwidth and passes the information to the video streaming encoder/decoder 149 to adjust the bitrate of a video encoder based on the feedback information. An adaptive intra-refresh module 146 sets a refresh frequency of constrained intra macroblocks that is adapted based on a set of factors. A periodic movement multiple reference frames module 158 estimates motion for several previous frames. A N-interleaved vertical intra refresh module 150 horizontally interleaves vertical line by line to reduce peak bandwidth requirements.

The individual enhancement modules 144, 146, 148, and 150 each provide different enhancements that aid in providing a live video stream of medical images. It will be understood that in a commercial product the entire suite of modules 144, 146, 148, and 150 may be used in combination. Alternatively a commercial product may include a smaller subset of modules 144, 146, 148, and 150, such as one, two, or three out of the four modules 144, 146, 148, and 150. Additionally, while an exemplary application is for live streaming of medical images, it will be understood that other non-medical applications are contemplated and within the scope of embodiments of the present invention. Moreover, it will be understood that the modules 144, 146, 148, and 150 may be selectively enabled/disabled based on the relative benefits to using the modules versus the computational overhead. It will also be understood that in a commercial embodiment a video streaming encoder/decoder application may include one or more of the modules 144, 146, 18, and 150. It will also be understood that in a commercial embodiment a receiving node also includes features, such as reporting tools, to support duplex operation.

It will also be understood that the modules 144, 146, 148, and 150 may be selectively used for high entropy content images in order to achieve a live video stream satisfying the standard required for medical images. In medical imaging the peak signal noise ratio (PSNR) of the images generally has to be high, even if the images are noisy, in order to achieve an acceptable image quality under the Just Noticeable Difference (JND) standard for compression of medical images. Investigations by the inventors indicated that a PSNR of between 37 to 39 is required to satisfy the JND standard for high entropy content medical images, with a PSNR of at least 38 being preferred.

### Adaptive Intra Refresh (Air)

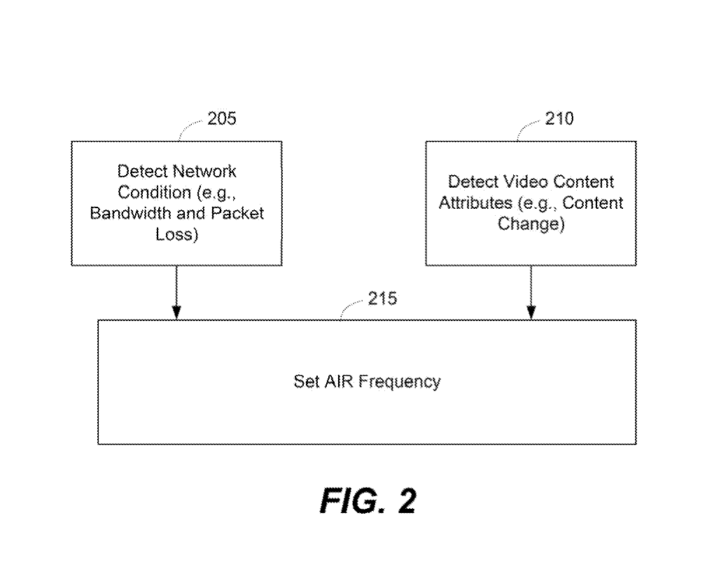

Aspects of Adaptive Intra Refresh (AIR) module 146 will now be described in greater detail. Referring to FIG. 2, network conditions are detected in block 205. The video content is detected in block 210. The AIR frequency is set in block 215, and additional randomization of the intra blocks may be performed.

In a telemedicine application with real time video streaming and low latency it is not practical to request duplicate packets via retransmission. This is particularly true for a duplex session in which there is a live interaction between doctors/clinicians during a telemedicine session. Additionally, video pre-filtering and post-processing cannot be performed for medical images because of the risk of generating a false medical diagnosis. If a video packet is either late or is lost, then the entire slice is lost for that frame, where a slice is a group of macroblocks. Additionally, loss of an entire slice of a frame results in a prediction error in the P frames that follow it. To address this problem, constrained intra macroblocks (MBs) (I-blocks) are regularly sent to refresh the video frame. The intra refresh period may, for example, have a default value of 150 frames, which would be 5 seconds at a frame rate of 30 frames per second

However, sending the constrained intra MBs in a frame consumes extra bandwidth. Thus, there is a compromise between the video quality and the number of constrained intra MBs per frame.

In one embodiment a frequency of constrained intra MBs is set that is based on network conditions and the video content. The following four factors may be used independently, in subsets, or together in combination:
1. Packet Loss: In one embodiment the AIR frequency is increased if packet loss increases.
2. Available Bandwidth: In one embodiment the AIR frequency is dependent on the available bandwidth. If the bandwidth is reduced, then the AIR may also be reduced proportionately.
3. Content. In one embodiment the AIR frequency may also be dependent on the video content type (low entropy or high entropy, which affects predictability) and whether there any sudden changes to the content that affects predictability. Examples of content change include sudden changes to texture, motion, brightness, and contrast. For example, in the case of ultrasound imaging, the content has a high entropy content. Additionally, sudden changes in content may occur in attempting to adjust an ultrasound probe to image moving organ/tissues, such as a beating heart. In one embodiment the type of content and content changes are monitored. If constrained intra is more efficient relative to predicted macroblocks then the AIR frequency is increased.
4. Intra period: In one embodiment the contained MBs refresh the whole frame in one intra period in a randomized fashion. That is, the constrained intra macroblocks (MBs) (I-blocks) are regularly sent to refresh the video frame with a random distribution of I blocks in a frame in the one intra period.

### Network Aware Rate Control Algorithm

Aspects of network aware rate control module 144 will now be described in greater detail. In one embodiment a network aware rate control is utilized. The network conditions are detected and the bitrate of a video rate controller is adjusted based on the feedback information.

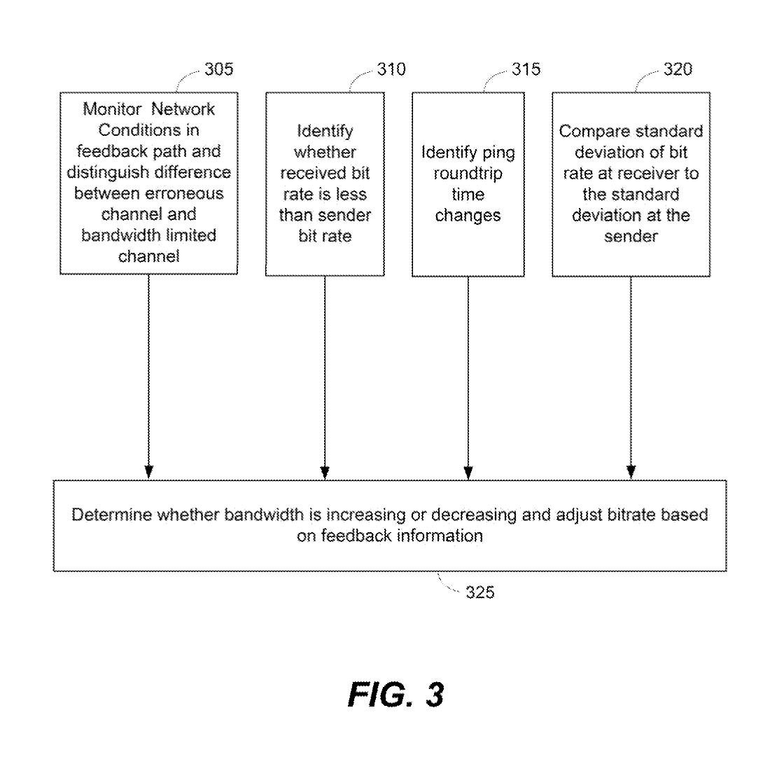

FIG. 3 illustrates a method in accordance with an embodiment of the present invention. In block 305 network conditions are monitored in a feedback path to distinguish differences between an erroneous channel and a bandwidth limited channel. This may include identifying whether the received bit rate is less than the sender bit rate in block 310. Ping roundtrip time changes may be identified in block 315. The standard deviation of a bit rate at the receiver may be compared the standard deviation at the sender (or to a threshold deviation) in block 320. From these determinations, the method may then determine whether and how to adjust the bitrate based on the feedback information in block 325.

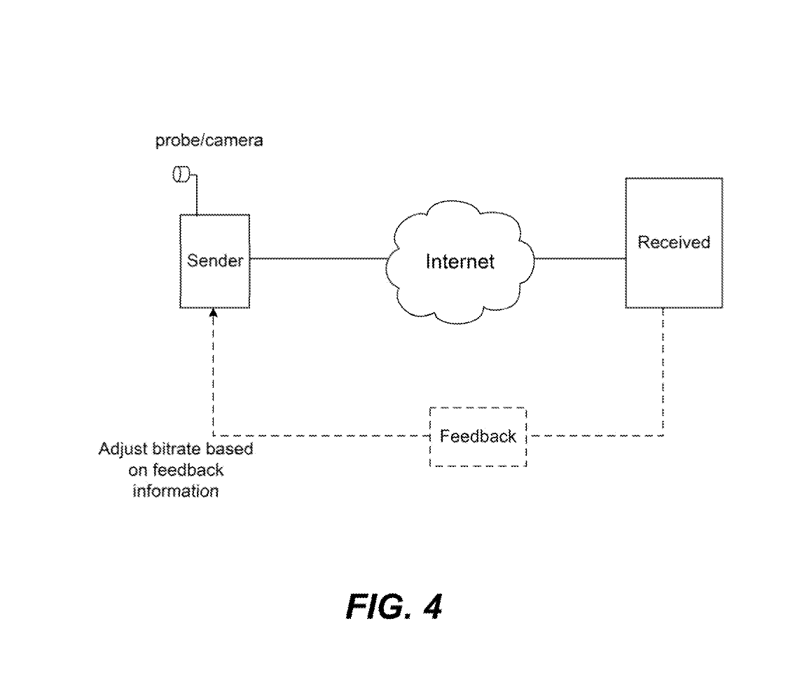

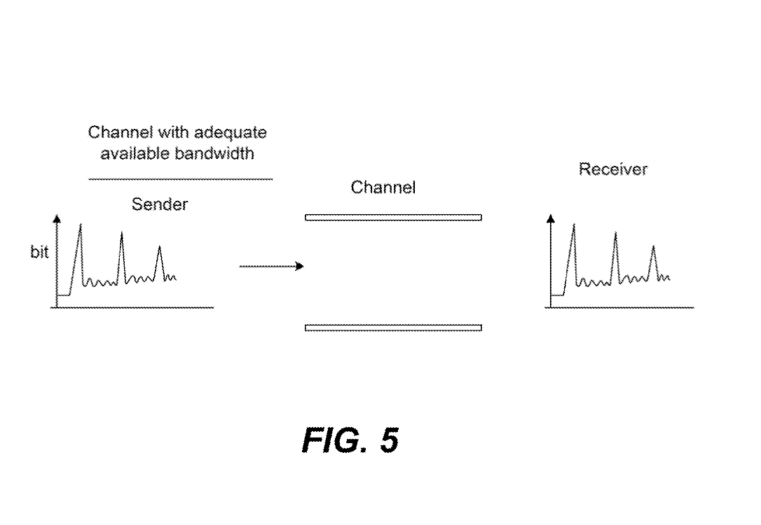

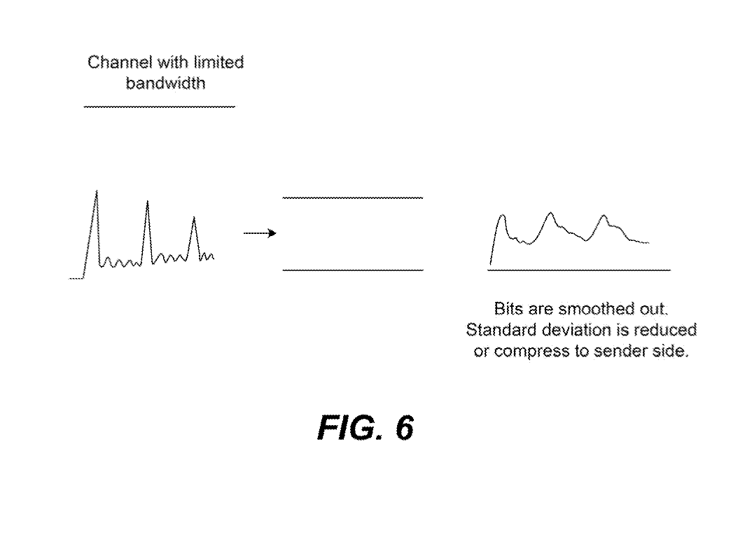

FIG. 4 illustrates the feedback path between the receiver and the sender. FIG. 5 and FIG. 6 illustrates aspects related to standard deviation. FIG. 5 illustrates bits sent by the sender have some time varying distribution. Because the channel has adequate bandwidth, the bits received at the receiver will have a nearly identical distribution of bits versus time. FIG. 6 illustrates how in a channel with limited bandwidth that the bits at the receiver will have a reduced standard deviation.

In one embodiment the feedback information differentiates between packet loss errors (an erroneous channel) and a bandwidth limited channel. In a bandwidth limited channel the received bitrate is less than the sender bitrate over a period of time. In contrast, in an erroneous channel, packet loss occurs at all bitrates.

In one embodiment a reduction is bandwidth is detected by the following:
1) By determining if the received bitrate is less than the sender bitrate. If the client received bitrate is less than the sender bitrate over a period of time, then the bandwidth has been reduced. For this situation, the sender can reducing the sending bitrate.
2) An increase in ping roundtrip time is an indication of network congestion. In particular, if there is a sudden rise in ping roundtrip time, it is an indication of congestion. As an example, an increase in ping roundtrip time above a pre-selected factor (e.g., an at least double rise in ping roundtrip time) may be used an indicator of congestion. If there is an increase in congestion then the target bitrate may be decreased on the encoder side at the sender node.

An increase in bandwidth can be sensed by several indicators:
1) If the channel has limited bandwidth, the bits at the receiver are smoothed out and thus the standard deviation, at the receiver side, is less then at the sender side. Consequently, a low standard deviation (below a pre-selected threshold) is an indication of a saturated channel with limited bandwidth. Conversely, if the received bit rate has a standard deviation above a threshold level, it indicated that the channel has more capacity than the current bitrate. FIG. 6 illustrates how in a channel with limited bandwidth the bits are initially transmitted at the sending node with a first standard deviation. However, at the receiving node the bits are smoothed out and the standard deviation is reduced as compared to the sender side.
2) Higher ping time indicates that either the channel is saturated or the processor is too busy. Lower ping times indicate that both processing power and the network bandwidth are available and that the bitrate of the video streaming application can be increased.

### Multiple Reference Frames

The periodic movement reference frame module 148 leverages off of the multiple reference frames features used in the H.264/AVC video codec standard originally developed for conventional video.

The H.264 standard allows a video encoder to choose among more than one previously decoded frame on which to base each macroblock in the next frame. H.264 supports up to 16 concurrent reference frames. Encoding multiple reference frames increases encoding time, which is one of the reasons that the multiple reference frames feature of H.264 is not commonly used. Additionally, even when the multiple reference frames feature of H/264 is used, only a small number of reference frames are used. In conventional video applications frames farther back in time have less correlation with the current frame. Moreover, in conventional video applications the frames are highly compressible. Thus, in conventional video applications there is typically little benefit to using the multiple reference frames feature of H.264 and even then only a small number of reference frames are used because of the high computational overhead and the low correlation with older frames.

Unlike the prior art, the present application of multiple reference frames is directed to the particular problems of streaming high entropy content medical images in a network having limited bandwidth. This results in a set of conditions in which the inventors have recognized that the use of multiple reference frames provides a significant improvement in compressibility.

High entropy content medical images have low compression ratios compared with conventional video images. That is the high noise content makes it difficult to achieve a high compression ratio for an ultrasound medical image without loss. Identifying additional techniques to increase compression without loss thus provides a significant advantage in a telemedicine environment in which network bandwidth is limited.

Additionally, in many medical applications there is a periodic movement in the frames associated with the circulatory system, such as the beating of the heart and the pulse of the blood. There may also be a periodic movement associated with the respiratory system if the breathing is rhythmic. This periodic movement increases the correlation with older frames. In the present invention, multiple reference frames are selectively employed only for: 1) high entropy content medical images, such as ultrasound images; and 2) for medical images in which there is biological rhythm. For example, the periodic pulsation of a patient's heart and the resultant pulsation of blood creates a periodic pulsation of blood in tissues being imaged. Similarly, the movement of air as a patient breathes can also result in a periodic movement of the diaphragm and lungs. In one embodiment 16 to 64 reference frames are utilized, with 20 being a preferred number of reference frames. That is, the number of reference frames for a high entropy medical image having an underlying periodic biological rhythm is greater than what is used for conventional video.

Calculations by the inventors indicate that the compression ratio for ultrasound images, Doppler ultrasound images, or other high entropy content medial images, may be increased by at least 25%. This is significant in view of the fact that it is difficult to compress high entropy content medical images with a high compression ratio. Thus when network bandwidth is limited this extra 25% increase in compression provides a substantial benefit.

It will be understood that the multiple reference frames feature may be used selectively for high entropy content medical images. That is, this feature does not have to be used for conventional video conferencing features, such as sending a video stream containing conventional video camera images of the doctors. Thus, it will be understood that the multiple reference frames feature may be enabled/disabled based on whether or not the video that is being streamed contains high entropy content medical images, such as ultrasound medial images.

Additionally, it will be understood that this feature may be selectively utilized for certain bandwidth conditions. When network bandwidth is constrained it may be necessary to increase compression of high entropy content medical images in order to maintain a live video stream. Thus, in some embodiments the multiple reference frames features is enabled when bandwidth is at or below a threshold level.

### N-Interleaved Vertical Intra Refresh

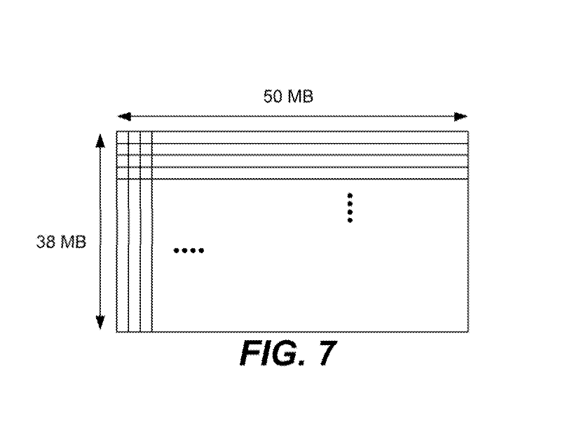

Aspects of the N-interleaved Vertical Intra Refresh module 150 will now be described. FIG. 7 shows a frame organized into a sequence of vertical bar macroblocks. In one embodiment of vertical intra refresh, intra MBs are sent as vertical bars. The period is set using the intra refresh period but won't exceed the number of MB columns in the frame. In the example of FIG. 7 the video resolution is 800×608, corresponding to 50 MB×38 MB. Thus there are 50 MB columns, with each column having 38 MBs.

A vertical intra refresh completes in (N-1) frames, where N is the width of the video frame in macroblocks. As an illustrative example, in a first frame col 0 and col 1 are intra MB columns In a second frame, columns 1 and 2 are intra MB columns. In a third frame, columns 2 and 3 will be intra MB columns. The process continues on with each frame so that in a 49^{th} frame, columns 48 and 49 are intra MB columns.

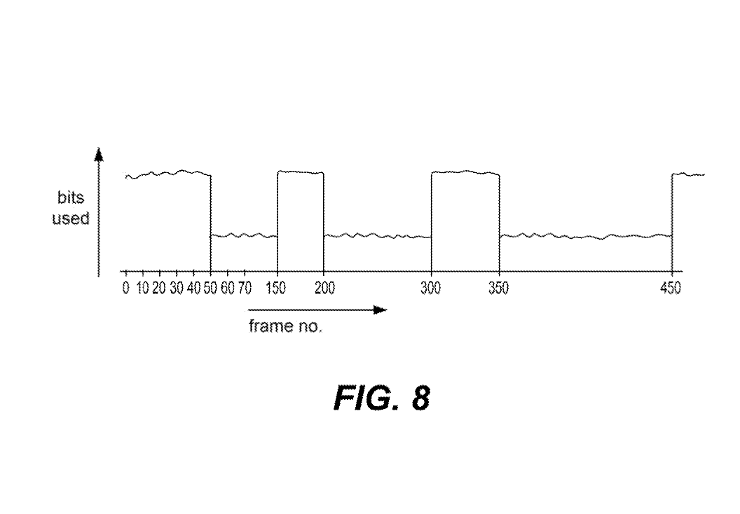

Suppose that the intra refresh period is 150 frames, which would be 5 seconds at a frame rate of 30 frames per second. Then frame 50 to frame 149 will not have any intra MB columns. Referring to FIG. 8, intra MBs take more bits that inter MBs, especially for static/linear motion content. That is, the bit rate will be very high for frames 0 to 49, then the bit rate will be reduced for frames 50 to 149, and so on. This is not ideal for real time streaming with constant bandwidth.

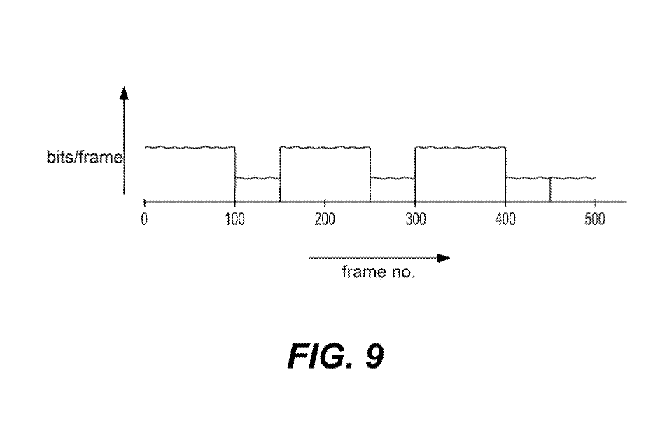

One way to improve the utilization of the available bandwidth is to n-interleave the video frame horizontally, thus increasing the width by “n” and then decreasing the height by “n”, which is illustrated in FIG. 9. By decreasing the height, the number of intra MB s will be reduced and bits used by that frame will be reduced. As an illustrative example, for 800×608, suppose that there is an interleave by “2” then it becomes 1600×304, which is 100×19 in MBs. For the same intra refresh period of 150, Intra MB columns will be sent in column 0 to 99, the bits per fames will be as illustrated in FIG. 9. The process may be continued to effectively eliminate fluctuations in bandwidth caused by vertical intra bars. For the same refresh period of 150, the process may, for example, be performed with an interleave by 3.

While the invention has been described in conjunction with specific embodiments, it will be understood that it is not intended to limit the invention to the described embodiments. On the contrary, it is intended to cover alternatives, modifications, and equivalents as may be included within the spirit and scope of the invention as defined by the appended claims. The present invention may be practiced without some or all of these specific details. In addition, well known features may not have been described in detail to avoid unnecessarily obscuring the invention. In accordance with the present invention, the components, process steps, and/or data structures may be implemented using various types of operating systems, programming languages, computing platforms, computer programs, and/or general purpose machines. In addition, those of ordinary skill in the art will recognize that devices of a less general purpose nature, such as hardwired devices, field programmable gate arrays (FPGAs), application specific integrated circuits (ASICs), or the like, may also be used without departing from the scope and spirit of the inventive concepts disclosed herein. The present invention may also be tangibly embodied as a set of computer instructions stored on a computer readable medium, such as a memory device.

What is claimed is:

1. A method for performing adaptive Intra Refresh (AIR) to refresh individual video frames of a video stream containing medical images in which there is a compromise between video quality and the number of constrained intra macroblocks per frame, comprising:
monitoring network conditions between a sending node and a receiving node;
detecting changes in video content of a frame to be sent and determining an efficiency of utilizing constrained intra macroblocks relative to predicted macroblocks; and
setting a frequency of constrained intra macroblocks of a transmitted video stream based on the monitored network conditions and the detected changes in video content.

2. The method of claim 1, wherein monitoring network conditions includes determining a packet loss and setting the frequency of constrained intra macroblocks to increase the frequency to adapt to increasing packet loss.

3. The method of claim 1, wherein monitoring network conditions includes determining an available bandwidth between the sending node and the receiving node and setting the frequency of constrained intra macroblocks includes reducing the frequency based on the available bandwidth.

4. The method of claim 1, wherein detecting changes in video content of individual frames includes detecting a change in at least one of texture, motion, brightness and contrast.

5. The method of claim 1, wherein the video content includes a high entropy content medical image.

6. The method of claim 5, wherein the video content includes an ultrasound medical image.

7. The method of claim 1 wherein the constrained intra macroblocks are sent in a random order to refresh a frame in one intra period.

8. A method of video streaming of ultrasound medical images, comprising:
utilizing multiple reference frames to encode an ultrasound medical image having a periodic biological rhythm.

9. The method claim 8, wherein the periodic biological rhythm is associated with the periodic flow of blood in a patient.

10. The method of claim 8, wherein sixteen to sixty-four reference frames are utilized for the multiple reference frames.

11. The method of claim 8, wherein multiple reference frames are used selectively for ultrasound medical images.

12. A method for performing variable bit rate video encoding of medical images, comprising:
providing a bit allocation rate control feedback path between a receiving node and a sending node to indicate network conditions based on ping roundtrip time, sending bit rate, and received bitrate; and
adapting a bit rate of a variable bit rate video encoder to encode a video stream of medical images based on network conditions.

13. The method of claim 12, wherein providing a bit allocation rate control feedback includes detecting a reduction in bandwidth.

14. The method of claim 13, wherein detecting a reduction in bandwidth includes detecting when the client received bitrate is less than the sending bitrate and in response adapting the bit rate to reduce the sending bitrate.

15. The method of claim 13, wherein detecting a reduction in bandwidth includes detecting a rise in a ping roundtrip time indicative of congestion and in response adapting the bit rate to decrease the sending bitrate.

16. The method of claim 12, wherein providing a bit allocation rate control feedback path includes detecting an increase in bandwidth.

17. The method of claim 16, wherein detecting an increase in bandwidth comprises monitoring a standard deviation of a received bitrate relative to a standard deviation of a sending bitrate and determining if the standard deviation of the received bitrate is indicative of a saturated channel.

18. The method of claim 17, wherein if the received bitrate has a standard deviation above a threshold value a determination is made that the channel has more capacity than the current bitrate.

19. The method of claim 16, wherein detecting an increase in bandwidth comprising detecting an attribute of ping time indicative of an increase in bandwidth and available processing power such that eh sending bitrate may be increased.

20. The method of claim 19, wherein a reduction in ping time below a pre-selected threshold is detected.

21. A method of improving utilizing of bandwidth to stream video, comprising:
interleaving vertical intra macroblocks horizontally across a set of frames having inter macroblocks, the interleaving being performed within an intra refresh period.

22. The method of claim 2l wherein the interleaving is performed so that there is a uniform distribution, at a frame level, of intra macroblocks.

23. The method of claim 21 wherein the interleaving is performed to distribute a total number of bits for intra macroblocks over frames within the intra refresh period to reduce a maximum number of bits per frame.

24. The method of claim 23, wherein a default sequence of intra macroblocks is interleaved to increase its width by n and decrease a bit height by n, where n is an integer.

25. A system for enhancing a live video stream of high entropy content medical images having a periodic biological rhythm, comprising:
a computing device having a processor and a memory hosting a video application;
a network aware rate control module to adapt a bit rate of a variable bit rate video encoder to encode the video stream of medical images based on network conditions;
an adaptive intra refresh module to set a frequency of constrained intra macroblocks of a transmitted video stream in a frame based on monitored network conditions and the detected changes in video content;
a periodic movement multiple reference frame module utilizing multiple reference frames to encode high entropy content medical image having a periodic biological rhythm; and
an intra refresh module to interleave vertical intra macroblocks horizontally across a set of frames having inter macroblocks, the interleaving being performed within an intra refresh period.
